(19) United States
(12) Patent Application Publication
(10) Pub. No.: US 2006/0234362 A1
(43) Pub. Date: Oct. 19, 2006

(54) Enzymatic method of making 1,2-diol derivatives and their esters with succine anhydride

(76) Inventors: Soon Ook Hwang, Daejeon (KR); Do Hoon Kim, Daejeon (KR); Hye Youn Ryu, Daejeon (KR); Tae Im Lee, Daejeon (KR); Sun Ho Chung, Daejeon (KR)

Correspondence Address: IPLA P.A., 3580 WILSHIRE BLVD., 17TH FLOOR, LOS ANGELES, CA, 90010, US

(21) Appl. No.: 10/558,057

(22) PCT Filed: Jun. 2, 2004
(86) PCT No.: PCT/KR04/01313

§ 371 Date: Nov. 22, 2005

(30) Foreign Application Priority Data
Jun. 3, 2003 (KR) 10-2003-0035470
Jun. 2, 2004 (WO) PCT/KR04/01313

(51) Int. Cl. C12P 7/02

(52) U.S. Cl. 435/155

(57) ABSTRACT

The present invention relates to a new process for the preparation of optically active alcohols represented by the general formula 2 and their esters represented by the general formula 3 in scheme 1. In more detail, this invention relates to the process for the preparation of optically active alcohols and their esters which are used as pharmaceutical intermediates by reacting the hydroxyl group stereospecifically by lipase after adding racemic alcohols represented by the general formula 1 and succinic anhydride as an acylating agent to the organic solvent. According to this invention, the primary hydroxyl group of 1,2-diols is transformed by other functional group and the secondary hydroxyl group is esterified stereospecifically with succinic anhydride as an acylating agent. Optically active alcohols and their esters of high optical purity in high yield can be produced by using succinic anhydride as an acylating agent because alcohols can be separated from their esters more easily than those of other conventional methods.

### TECHNICAL FIELD

The present invention relates to a new process for the easy preparation of optically active alcohols and their esters by reacting the hydroxy group of racemic 1,2-diol derivatives represented by the general formula 1 stereospecifically after adding succinic anhydride as an acylating agent and lipases as biocatalysts to organic solvents.

This invention relates to a process for preparing the products of high optical purity in high yield by separating alcohols from their esters easily after reaction using succinic anhydride as an acylating agent. Racemic alcohols represented by the general formula 1 in scheme 1 are composed of (S)-alcohols and (R)-alcohols respectively and they are used as intermediates in preparing important pharmaceuticals.

There are some enzymatic methods to prepare optically active 1,2-diols. In most cases, the primary hydroxyl group is transformed by other functional group and the secondary hydroxyl group is hydrolyzed or esterified stereospecifically.

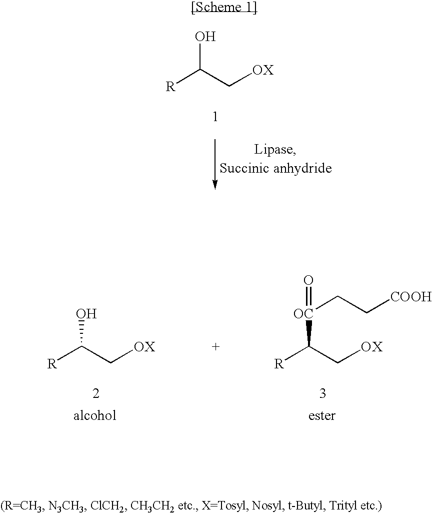

### BACKGROUND ART

Hamaguchi et al. obtained (S)-2-hydroxy-3-chloropropyl p-toluenesulfonate (99% ee) and (R) 2-acetoxy-3-chloropropyl p-toluenesulfonate (99% ee) by the hydrolysis of racemic 2-acetoxy-3-chloropropyl p-toluenesulfonate using LPL (Amano) as biocatalyst (see Agric. Biol. Chem., 50(2), 375-380(1986)).

On the other hand, Kim and Choi obtained (R)-2-hydroxy-3-chloropropyl tritylate (yield 54%, 72% ee) and (S)-2-acetoxy-3-chloropropyl tritylate (yield 43%, 98% ee) by the transesterification of 2-hydroxy-3-chloropropyl tritylate using PS lipase and vinyl acetate as the acylating agent in toluene (see J. Org. Chem., 57: 1605-1607(1992)).

Recently, the primary hydroxyl group of 1,2-diol derivatives was transformed by tosyl group and the secondary hydroxyl group was esterified stereospecifically (International Application No. PCT/KR2004/001005).

However, these processes have common problems in separating alcohols from their esters respectively on the large scale.

For solving this problem, Fiaud et al. (Tetrahedron Letter, vol. 33: 6967-6970(1992)) and Gutman et al. (Tetrahedron: Asymmetry, vol. 4: 839-844(1993)) obtained (−)-tert-butyl cyclobutylidenethanol (89% ee) and (S)-1-phenylethanol (100% ee) on the large scale by the solvent extraction using succinic anhydride as an acylating agent.

Thus, alcohols and their esters of high optical purity could be produced easily by easy separation of alcohols from their esters after reaction when succinic anhydride was used as an acylating agent.

### DISCLOSURE OF INVENTION

Instead of Chen's method using isopropenyl acetate as an acylating agent (J. Chem. Soc. Perkin Trans 1, 2559-2561(1990)), in the present invention, succinic anhydride was used as an acylating agent in the reaction and the easy recovery of alcohols and their esters was possible by solvent extraction after reaction. Thus, the process of preparing optically active alcohol derivatives and esters of high optical purity in high yield was developed.

Accordingly, the objective of this invention is to provide the method of preparing optically active alcohols and their esters of high optical purity in high yield by recovering the products easily after reaction using succinic anhydride as an acylating agent.

For the above objective, this invention consists of the process for reacting racemic alcohol represented by the general formula 1 stereospecifically by lipase using succinic anhydride as an acylating agent in organic solvent.

This invention is explained in more detail as follows.

As mentioned above, in this invention, racemic alcohol represented by the formula 1 and succinic anhydride were placed in the organic solvent. Then, lipase was added to the mixture. The reaction was carried out in order to make optically active alcohols and their esters as shown in scheme 1.

For the lipase, commercially available ones and, if necessary, home-made ones can be used. Non-limiting examples of the commercially available lipase include Novoyme 435 from Novo Ltd. and those manufactured by Amano Inc. such as PS, PS-D, PS-C and AK lipase.

After reaction, optically active alcohols and their esters are separated by known methods such as solvent extraction, crystallization and so on.

Optically active 2-hydroxy-3-azidopropyl t-butylate was determined by a gas chromatography (Donam Instruments Inc. Model DS 6200) equipped with chiral column (Chiraldex B-PM, Alltech). The oven temperature was maintained initially at 100° C. for 10 min and then raised at the rate of 0.5° C./min to 160° C., and maintained for 3 minutes. The typical retention time of the components in this invention was as follows:
(S)-2-hydroxy-3-azidopropyl t-butylate—13.1 min
(R)-2-hydroxy-3-azidopropyl t-butylate—14.1 min

Optically active 2-hydroxypropyl p-toluenesulfonate was determined by a HPLC (Lab Alliance Inc. Model 201) equipped with chiral column (Chiralcel OB-H, Daicel) using hexane and isopropyl alcohol mixture (80:20) as mobile phase. The Absorbance was 220 nm and flow rate was 0.65 ml/min. The typical retention time of the components in this invention was as follows:
(S)-2-hydroxypropyl p-toluenesulfonate—20 min
(R)-2-hydroxypropyl p-toluenesulfonate—26 min

Analytical condition of optically active 2-hydroxy-3-chloropropyl p-toluenesulfonate was the same as that of 2-hydroxypropyl p-toluenesulfonate. The typical retention time of the components in this invention was as follows:
(S)-2-hydroxy-3-chloropropyl p-toluenesulfonate—31 min
(R)-2-hydroxy-3-chloropropyl p-toluenesulfonate—41 min

Analytical condition of optically active 2-hydroxybutyl p-toluenesulfonate was the same as that of 2-hydroxypropyl p-toluenesulfonate except that flow rate was 0.45 ml/min. The typical retention time of the components in this invention was as follows:
(S)-2-hydroxybutyl p-toluenesulfonate—24.9 min
(R)-2-hydroxybutyl p-toluenesulfonate—27.9 min

Analytical condition of optically active 2-hydroxy-3-chloropropyl 3-nitrobenzensulfonate was the same as that of 2-hydroxypropyl p-toluenesulfonate. The typical retention time of the components in this invention was as follows:
(R)-2-hydroxy-3-chloropropyl 3-nitrobenzensulfonate—58.7 min
(S)-2-hydroxy-3-chloropropyl 3-nitrobenzensulfonate—63.7 min

Optically active 2-hydroxypropyl tritylate was determined using chiral column (Chiralcel OJ-H, Daicel) and hexane and isopropyl alcohol mixture (95:5) as mobile phase. And flow rate was 0.7 ml/min. The typical retention time of the components in this invention was as follows:
(S)-2-hydroxypropyl tritylate—17 min
(R)-2-hydroxypropyl tritylate—24 min

And racemic 2-hydroxy-3-azidopropyl t-butylate, 2-hydroxypropyl p-toluenesulfonate, 2-hydroxy-3-chloropropyl p-toluenesulfonate, 2-hydroxybutyl p-toluenesulfonate, 2-hydroxy-3-chloropropyl 3-nitrobenzenesulfonate and 2-hydroxypropyl tritylate thus synthesized were confirmed by FT-NMR (Burker Inc., Model DPX300) respectively and the results are as follows:

2-hydroxy-3-azidopropyl t-butylate:

¹H-nuclear magnetic resonance (¹H-NMR)(CDCl₃):

δ 3.75(m, 1H), 3.23 to 3.29(m, 4H), 1.09(s, 9H)

2-hydroxypropyl p-toluenesulfonate:

¹H-nuclear magnetic resonance (¹H-NMR)(CDCl₃):

δ 7.72(d, 2H), 7.28(d, 2H), 3.78 to 3.97(m, 3H), 3.06(bs, 1H), 2.38(s, 3H), 1.09(d, 3H)

2-hydroxy-3-chloropropyl p-toluenesulfonate:

¹H-nuclear magnetic resonance (¹H-NMR)(CDCl₃):

δ 7.8(d, 2H), 7.4(d, 2H), 3.61 to 4.15(m, 3H), 2.48(s, 3H), 1.27(d, 2H)

2-hydroxybutyl p-toluenesulfonate:

¹H-nuclear magnetic resonance (¹H-NMR)(CDCl₃):

δ 7.8(d, 2H), 7.39(d, 2H), 3.78 to 4.08(m, 3H), 2.47(s, 3H), 1.46 to 1.51(m, 2H), 0.92 to 0.97(t, 3H)

2-hydroxy-3-chloropropyl 3-nitrobenzenesulfonate:

¹H-nuclear magnetic resonance (¹H-NMR)(CDCl₃):

δ 7.7 to 8.1(m, 4H), 3.7 to 4.2(m, 3H), 1.22(d, 2H)

2-hydroxypropyl tritylate:

¹H-nuclear magnetic resonance (¹H-NMR)(CDCl₃)

δ 4.02(m, 1H), 3.02 to 3.2(dq, 2H), 2.47(bs, 1H), 1.16(d, 3H)

A better understanding of the present invention may be obtained through the following examples which are set forth to illustrate, but are not to be construed as the limit of the present invention.

### EXAMPLE 1

t-butyl glycidyl ether (0.5 g) was dissolved in the mixture of ethanol and distilled water. NH₄Cl (0.41 g), NaOH (0.153 g) and NaN₃(0.5 g) were added to the mixture respectively. Then the reaction was carried out for 2 hours at 80° C. After reaction, the reaction mixture was extracted with water and dichloromethane. The combined organic phase was dried over and 2-hydroxy-3-azidopropyl t-butylate was obtained and confirmed by FT-NMR.

Succinic anhydride (0.5 g) and t-butylmethylether (5 ml) were placed in a 15 ml vial. Then, racemic 2-hydroxy-3-azidopropyl t-butylate (0.05 g) and PS lipase (0.2 g) were added to the mixture. The reaction was carried out for 111 hours at 45° C. and (S)-2-hydroxy-3-azidopropyl t-butylate (98.3% ee) was obtained at 52.1% conversion. (R)-3-azido-2-O-succinyl)-propyl t-butylate were extracted with Na₂CO₃ aqueous solution and the mixture were hydrolyzed by NaOH. (R)-2-hydroxy-3-azidopropyl t-butylate (89.6% ee) were obtained.

### EXAMPLE 2

1,2-Propanediol (7.6 ml) was dissolved in dichloromethane (50 ml) at room temperature, 4-dimethylaminopyridine (0.49 g) and p-toluenesulfonyl chloride (24.7 g) were added to it respectively and maintained at 0˜5° C. And triethylamine (13.2 ml) was added slowly for 1 hour under nitrogen atmosphere. Then the reaction was carried out for 24 hours at room temperature. After reaction, the reaction mixture was poured into an ice-water mixture and extracted with dichloromethane. The combined organic phase was dried over and 2-hydroxypropyl p-toluenesulfonate (17.3 g, yield 73%) was obtained and confirmed by FT-NMR.

Succinic anhydride (0.1 g) and t-butylmethylether (5 ml) were placed in a 15 ml vial. Then, racemic 2-hydroxypropyl p-toluenesulfonate (0.05 g) and PS lipase (0.2 g) were added to the mixture. The reaction was carried out for 22 hours at 45° C. and (S)-2-hydroxypropyl p-toluenesulfonate (99.0% ee) was obtained at 54.4% conversion.

### EXAMPLE 3

Instead of 1,2-propanediol, 3-chloro-1,2-propanediol (11.05 ml) was dissolved in dichloromethane (50 ml), and the synthesis was performed as shown in Example 2.

After synthesis, 2-hydroxy-3-chloropropyl p-toluenesulfonate (22.3 g, yield 83%) was obtained and confirmed by FT-NMR.

2-Hydroxy-3-chloropropyl p-toluenesulfonate was used instead of 2-hydroxypropyl p-toluenesulfonate and PS-D lipase was used instead of PS lipase as shown in Example 2 and after 8 hours of reaction. (R)-2-hydroxy-3-chloropropyl p-toluenesulfonate (99.0% ee) were obtained at 47.1% conversion.

### EXAMPLE 4

Instead of 1,2-propanediol, 1,2-butanediol (2.25 ml) was dissolved in dichloromethane (50 ml) at room temperature, 4-dimethylaminopyridine (0.12 g) and p-toluenesulfonyl chloride (6.19 g) were added to it respectively and maintained at 0˜5° C. Triethylamine (3.28 ml) was added slowly for 1 hour under nitrogen atmosphere. Then the synthesis was performed as shown in Example 2.

After synthesis, 2-hydroxybutyl p-toluenesulfonate (4.95 g, yield 81%) was obtained and confirmed by FT-NMR.

2-hydroxybutyl p-toluenesulfonate was used instead of 2-hydroxypropyl p-toluenesulfonate as shown in Example 2 and after 67 hours of reaction, (S)-2-hydroxybutyl p-toluenesulfonate (99.0% ee) was obtained at 50.3% conversion.

### EXAMPLE 5

Instead of 1,2-propanediol, 3-chloro-1,2-propanediol (5.5 ml) was dissolved in dichloromethane (50 ml) at room temperature, 4-dimethylaminopyridine (0.12 g) and 3-nitrobenzenesulfonyl chloride (12.2 g) were added to it respectively and maintained at 0˜5° C. Triethylamine (3.29 ml) was added slowly for 1 hour under nitrogen atmosphere. Then the synthesis was performed as shown in Example 2.

After synthesis, 2-hydroxy-3-chloropropyl 3-nitrobenzenesulfonate (12.49 g, yield 82%) was obtained and confirmed by FT-NMR.

2-Hydroxy-3-chloropropyl 3-nitrobenzenesulfonate was used instead of 2-hydroxypropyl p-toluenesulfonate and PS-D lipase was used instead of PS lipase as shown in Example 2 and after 96 hours of reaction, (R)-2-hydroxy-3-chloropropyl 3-nitrobenzenesulfonate (16.1% ee) were obtained at 12.2% conversion.

### EXAMPLE 6

1,2-Propanediol (1 g) was dissolved in dichloromethane (10 ml) at room temperature, 4-dimethylaminopyridine (0.044 g) and triphenylmethyl chloride (2.78 g) were added to it respectively and maintained at 0˜5° C. Triethylamine (1.89 ml) was added slowly for 1 hour under nitrogen atmosphere. Then the synthesis was performed as shown in Example 2.

After synthesis, 2-hydroxypropyl tritylate was obtained and confirmed by FT-NMR.

2-Hydroxypropyl tritylate was used instead of 2-hydroxypropyl p-toluenesulfonate and CAL ipase was used instead of PS lipase as shown in Example 2 and after 33 hours of reaction, (S)-2-hydroxypropyl tritylate (99.0% ee) was obtained at 44.8% conversion. After reaction, (R)-2-O-succinylpropyl tritylate was extracted with Na₂CO₃ aqueous solution and the solution were hydrolyzed by NaOH. (R)-3-hydroxypropyl tritylate (95.7% ee) was obtained.

### EXAMPLES 7-9

Enzymatic transesterification of 2-hydroxypropyl p-toluenesulfonate was carried out using lipases as shown in Table 1 instead of PS lipase in Example 2. The results are shown in Table 1.

TABLE 1

| | | | | % ee for (S)-2- |
|---|---|---|---|---|
| | | Reaction | | hydroxypropyl p- |
| Example | Lipase | Time(hr) | Conversion(%) | toluenesulfonate |
| 7 | PS-D | 23 | 60.4 | 96.1 |
| 8 | CAL | 22 | 49.6 | 90.7 |
| 9 | PS-C | 29 | 56.5 | 91.0 |

### EXAMPLES 10-11

Enzymatic transesterification of 2-hydroxypropyl p-toluenesulfonate was carried out using the following solvents as shown in Table 2 instead of t-butylmethylether. The results are shown in Table 2.

TABLE 2

| | | | | % ee for (R)-2- |
|---|---|---|---|---|
| | Acylating | Reaction | | hydroxypropyl p- |
| Example | agent | time(hr) | Conversion(%) | toluenesulfonate |
| 10 | isopropyl- | 22 | 49.4 | 99.0 |
| | ether | | | |
| 11 | toluene | 27 | 50.8 | 99.0 |

### INDUSTRIAL APPLICABILITY

In accordance with this invention, the starting material can be synthesized at lower cost by simple method. With using succinic anhydride as an acylating agent, alcohols and their esters of high optical purity could be produced in high yield after enzymatic reaction. Therefore it is a very useful process on the industrial scale.

What is claimed is:

1. A process for preparing optically active alcohols represented by the general formula 2 and their esters represented by the general formula 3 from racemic alcohols represented by the general formula 1, whose secondary hydroxyl group is esterified stereospecifically by lipase with succinic anhydride as an acylating agent in the organic phase.

2. The process according to claim 1, wherein R in the general formula 1 is CH₃, N₃CH₂, CH₃CH₂, ClCH₂.

3. The process according to claim 1, wherein X in the general formula 1 is trityl, t-butyl, tosyl, nosyl.
